(19) United States
(12) Patent Application Publication
(10) Pub. No.: US 2024/0206982 A1
(43) Pub. Date: Jun. 27, 2024

(54) DEFLECTABLE CATHETER WITH LOCALIZATION SENSOR

(71) Applicant: Covidien LP, Mansfield, MA (US)

(72) Inventors: Franklin J. Burquest, Crystal, MN (US); Evan M. Gustafson, Sioux Falls, SD (US); Nathan J. Knutson, Long Lake, MN (US); Bethany A. Palmer, Coon Rapids, MN (US); Tina M. Berthiaume, Champlin, MN (US)

(21) Appl. No.: 18/288,983

(22) PCT Filed: May 21, 2022
(86) PCT No.: PCT/IB2022/054769

§ 371 Date: Oct. 30, 2023

Related U.S. Application Data
(60) Provisional application No. 63/194,703, filed on May 28, 2021.

(51) Int. Cl. A61B 34/20; A61B 1/005; A61B 1/267; A61B 5/06; A61M 25/01

(52) U.S. Cl. CPC A61B 34/20; A61B 1/0052; A61B 1/0055; A61B 1/0057; A61B 1/2676; A61B 5/062; A61B 2034/2051; A61M 25/0127; A61M 25/0136; A61M 25/0138; A61M 25/0147

(57) ABSTRACT

A deflectable catheter (200) for guiding surgical tools that are passed therethrough includes a tubular structure (215), a sensor coil (213) disposed around an end portion of the tubular structure and a pull wire assembly (211, 212), which is coupled to the end portion of the tubular structure and is configured to cause a distal portion of the tubular structure to deflect when the pull wire assembly is operated. The catheter further includes a coil wire (210) disposed around a portion of the tubular structure and configured to restrain the pull wire.

### FIELD

This disclosure relates to a catheter, and more particularly, to a deflectable catheter with a location sensor for navigating near to a target within a bronchial tree.

### BACKGROUND

A common interventional procedure in the field of pulmonary medicine is bronchoscopy, in which a bronchoscope is inserted into the airways through the patient's nose or mouth. In some instances, a catheter is inserted into a working channel of the bronchoscope so that airways smaller than those which the bronchoscope is capable of traversing can be navigated through. Diagnostic and therapy tools may be inserted through the catheter to biopsy a specimen or treat a previously diagnosed lesion.

An electromagnetic navigation bronchoscopy (ENB) system may be used to give users continuous virtual visualization of the alignment of the catheter tip during ENB. A location sensor is placed at the tip of the catheter to display alignment of the catheter tip to the user. However, there may not be a way to make any fine adjustments to the alignment of the catheter tip other than typical push and torque maneuvers.

### SUMMARY

One aspect of the disclosure is directed to a catheter navigation system. The catheter navigation system includes an electromagnetic field generator configured to generate an electromagnetic field and a deflectable catheter (EWC). The deflectable catheter includes a tubular structure, a sensor wire, a pull wire assembly, and a coil wire. The tubular structure receives and guides the navigation of a surgical tool. The sensor wire is disposed around an end portion of the tubular structure and senses the electromagnetic field. The pull wire assembly is coupled to the end portion of the tubular structure and causes a distal portion of the tubular structure to deflect when the pull wire assembly is operated. The coil wire is disposed around a portion of the tubular structure.

Implementations of this aspect of the disclosure may include one or more of the following features. The pull wire assembly may include a pull ring fixedly coupled to the end portion of the tubular structure and a pull wire coupled to the pull ring. The sensor wire may be positioned distal from the pull ring. The catheter may further include braided wire disposed around a portion of the flexible tubular structure. The coil wire may be disposed around the braided wire and the pull wire. The pull wire assembly may further include a lumen disposed between the coil and the braided wire, and configured to receive the pull wire. The catheter may include a sensor wire coupled to the sensor wire and disposed along a portion of the length of the catheter between the braided wire and the coil. The catheter may include a lumen disposed between the coil and the braided wire, and configured to receive the sensor wire.

### BRIEF DESCRIPTION OF THE DRAWINGS

Various aspects and embodiments of the disclosure are described hereinbelow with references to the drawings, wherein:

FIG. 1 is a depiction of an electromagnetic navigation (EMN) system in accordance with the disclosure;

FIG. 2 is a perspective view of an end portion of a deflectable catheter (EWC) in accordance with the disclosure;

FIG. 3A is a sectional view of a deflectable catheter in accordance with the disclosure;

FIG. 3B is a sectional view of the deflectable catheter of FIG. 3A taken along the section lines A-A;

FIG. 3C is a sectional view of detail B of the deflectable catheter of FIG. 3B; and

FIG. 4 is a side view of a handle assembly for the deflectable catheter of FIG. 2.

### DETAILED DESCRIPTION

This disclosure is directed to improvements to catheter systems which allow for fine adjustments to the alignment of a catheter tip with a target to enable an access tool to perform biopsy and/or therapeutic procedures more accurately and effectively in comparison to prior devices. The catheter system of this disclosure incorporates a distal pull ring and pull wire to add deflection capability to the catheter or catheter. The pull ring may be situated proximal of a distal sensor coil. According to this configuration, when a user pulls the pull wire, the distal tip deflects, which causes a change in alignment of the catheter tip that is visible to the user on a display using a navigation software system. The deflectable catheter may maintain the same inside diameter (ID) and outside diameter (OD) as an original catheter.

A wire coil, which is separate from the location sensor coil, may be wrapped around the shaft within the catheter wall at a distal end portion of the catheter over the pull wire to prevent the pull wire from tearing through the catheter wall when the pull wire is placed under tension. The handle of the catheter system may include a slider attached to the pull wire. Pulling the slider in a proximal direction causes the catheter tip to deflect. Releasing the slider allows the catheter tip to relax and straighten out. The handle slider may use two or more detents to hold the deflection of the catheter tip. The catheter system may be used to deflect a straight catheter or a pre-curved catheter.

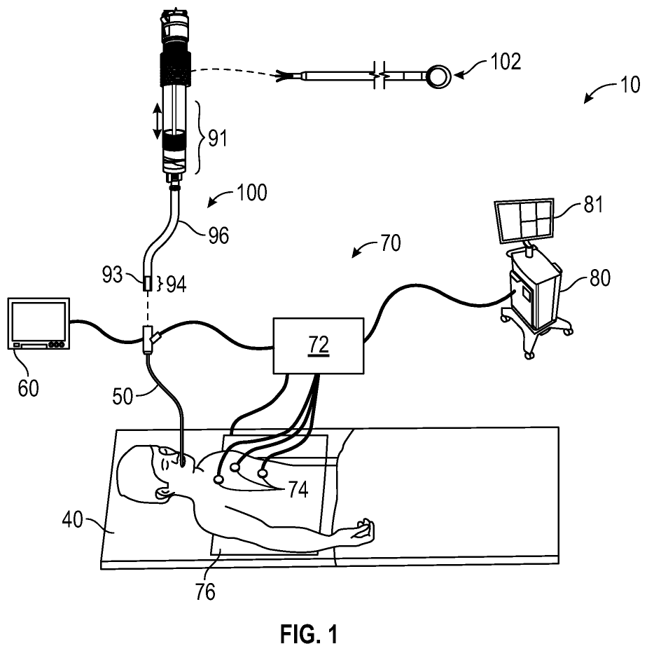

FIG. 1 shows an electromagnetic navigation (EMN) system 10 in accordance with the disclosure. One such EMN system is the ELECTROMAGNETIC NAVIGATION BRONCHOSCOPY® system currently sold by Medtronic PLC. Among other tasks that may be performed using the EMN system 10 are planning a pathway to a target, navigating a positioning assembly to the target, navigating a biopsy tool to the target tissue to obtain a tissue sample from the target tissue using the biopsy tool, digitally marking the location where the tissue sample was obtained, and placing one or more echogenic markers at or around the target.

EMN system 10 generally includes an operating table 40 configured to support a patient; a bronchoscope 50 configured for insertion through the patient's mouth into the patient's airways; monitoring equipment 60 coupled to bronchoscope 50, e.g., a video display for displaying video images received from the video imaging system of the bronchoscope 50; a tracking system 70 including a tracking module 72, reference sensors 74, and an electromagnetic field generator 76, which is also referred to as a transmitter mat; a workstation or computer 80 including software and/or hardware used to facilitate identification of a target, pathway planning to the target, navigation of a medical device to the target, and/or confirmation and/or determination of the placement of the catheter 96, or a suitable device therethrough, relative to the target.

Computer 80 may be any suitable computer including a storage medium and a processor, which is capable of executing instructions stored on the storage medium. Workstation 80 may further include a database configured to store patient data, CT data sets including CT images, fluoroscopic data sets including fluoroscopic images and video, fluoroscopic 3D reconstruction, navigation plans, and any other such data. Although not explicitly illustrated, Computer 80 may include inputs, or may otherwise be configured to receive, CT data sets, fluoroscopic images/video and other data described herein. Additionally, workstation 80 includes a display configured to display graphical user interfaces. Computer 80 may be connected to one or more networks through which one or more databases may be accessed.

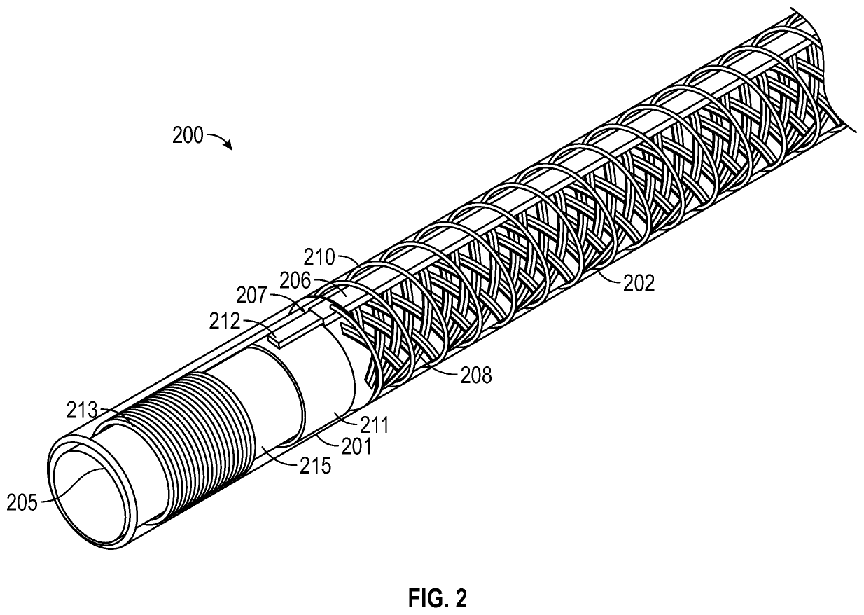

FIG. 1 depicts a catheter guide assembly 100, which is usable with the EMN system 10. The catheter guide assembly 100 includes a handle 91, which is connected to a catheter 96, such as the catheter 200 depicted in FIG. 2. In practice, the catheter 96 is placed into the working channel of a bronchoscope 50. The location of the EM sensor 94, and thus the distal end 93 of the catheter 96, within an electromagnetic field generated by the electromagnetic field generator 76, can be derived by the tracking module 72 and the computer 80. Catheter guide assembly 100 includes a handle 91 that can be manipulated to steer the distal tip 93 of the catheter 96.

As illustrated in FIG. 1, the patient is shown lying on operating table 40 with bronchoscope 50 inserted through the patient's mouth and into the patient's airways. Bronchoscope 50 includes a source of illumination and a video imaging system (not explicitly shown) and is coupled to monitoring equipment 60, e.g., a video display, for displaying the video images received from the video imaging system of bronchoscope 50.

Catheter guide assembly 100 includes catheter 96, which is configured for insertion through a working channel of bronchoscope 50 into the patient's airways (although the catheter guide assembly 100 may alternatively be used without bronchoscope 50). A six degrees-of-freedom electromagnetic tracking system 70 or any other suitable positioning measuring system, is utilized for performing navigation, although other configurations are also contemplated. Tracking system 70 is configured for use with catheter guide assembly 100 to determine and track the position and orientation of the EM sensor 94 as it moves in conjunction with the catheter 96 through the airways of the patient.

As shown in FIG. 1, an electromagnetic field generator 76, which may be in the form of a transmitter mat, is positioned beneath the patient. Electromagnetic field generator 76 and the reference sensors 74 are interconnected with tracking module 72, which derives the location of each reference sensor 74 in six degrees of freedom. Reference sensors 74 are attached to the chest of the patient. The six degrees of freedom coordinates of reference sensors 74 are sent to computer 80, which includes an application 81, which calculates a patient coordinate frame of reference based on sensor data output from the reference sensors 74.

Also shown in FIG. 1 is a catheter biopsy tool 102 that is insertable into the catheter guide assembly 100 following navigation of the catheter 96 to a target. The biopsy tool 102 is used to collect one or more tissue samples from the target tissue. The biopsy tool 102 may be further configured for use in conjunction with tracking system 70 to facilitate navigation of the biopsy tool 102 to the target tissue, tracking of a location of biopsy tool 102 as it is manipulated relative to the target tissue to obtain the tissue sample, and/or marking the location where the tissue sample was obtained.

Although navigation is detailed above with respect to EM sensor 94 being included in the catheter 96, it is also envisioned that EM sensor 94 may be embedded or incorporated within biopsy tool 102 where biopsy tool 102 may alternatively be utilized for navigation without need of the LG or the necessary tool exchanges that use of the LG requires. A variety of useable biopsy tools may be used with the EMN system 10 as described herein.

With respect to the planning phase, computer 80 utilizes previously-acquired computed tomography (CT) image data for generating and viewing a three-dimensional (3D) model or rendering of the patient's airways, enables the identification of a target on the 3D model (either automatically, semi-automatically, or manually), and allows for determining a pathway through the patient's airways to the target. More specifically, CT images acquired from previous CT scans are processed and assembled into a 3D volume, which is then utilized to generate the 3D model of the patient's airways. The 3D model may be presented on a display 81 associated with computer 80 or in any other suitable fashion. Using computer 80, various views of the 3D model or enhanced two-dimensional (2D) images generated from the 3D model may be presented. The enhanced two-dimensional images may possess some three-dimensional capabilities because they are generated from three-dimensional data.

The 3D model may be manipulated to facilitate identification of a target on the 3D model or 2D images, and selection of a suitable pathway through the patient's airways to access the target. The 3D model may also show marks of the locations where previous biopsies were performed, including the dates, times, and other identifying information regarding the tissue samples obtained. These marks may also be selected as the target to which a pathway can be planned. Once selected, the pathway plan, the 3D model, and the images derived therefrom can be saved and exported to a navigation system for use during the navigation phase. The ILLUMISITE software suite currently sold by Medtronic PLC includes one such planning software.

With respect to the navigation phase, EM sensor 94, in conjunction with tracking system 70, enables tracking of the catheter 96 and/or a tool, e.g., a biopsy tool 102, as the catheter 96 or tool 102 is advanced through the patient's airways. The position and orientation of a distal portion of the catheter 96 may be utilized for performing registration of the CT images and the pathway for navigation. Tracking system 70 includes the tracking module 72, reference sensors 74, and the transmitter mat 76. Tracking system 70 may be configured for use with catheter 96 and particularly EM sensor 94. Alternatively, locatable guide (not shown) and a sensor disposed at the distal portion of the locatable guide are configured for insertion through the catheter 96 into patient P's airways (either with or without bronchoscope 50) and may be selectively lockable relative to one another via a locking mechanism.

Transmitter mat 76 is positioned beneath patient P. Transmitter mat 76 generates an electromagnetic field around at least a portion of the patient P within which the position of reference sensors 74 and the EM sensor 94 can be determined with use of a tracking module 72. A second EM sensor may also be incorporated into the end of the tool 102. The second EM sensor may be a five degree-of-freedom sensor or a six degree-of-freedom sensor. One or more of reference sensors 74 are attached to the chest of the patient P. Registration is generally performed to coordinate locations of the three-dimensional model and two-dimensional images from the planning phase, with the patient P's airways as observed through the bronchoscope 50 and allow for the navigation phase to be undertaken with knowledge of the location of the sensor 94.

Registration of the patient P's location on the transmitter mat 76 may be performed by moving an EM sensor, e.g., EM sensor 94, through the airways of the patient P. More specifically, data pertaining to locations of EM sensor 94, while the locatable guide is moving through the airways, is recorded using transmitter mat 76, reference sensors 74, and tracking system 70. A shape resulting from this location data is compared to an interior geometry of passages of the three-dimensional model generated in the planning phase, and a location correlation between the shape and the three-dimensional model based on the comparison is determined, e.g., utilizing the software on the computer 80. In addition, the software identifies non-tissue space (e.g., air filled cavities) in the three-dimensional model. The software aligns, or registers, an image representing a location of sensor 94 with the three-dimensional model and/or two-dimensional images generated from the three-dimension model, which are based on the recorded location data and an assumption that locatable guide remains located in non-tissue space in patient P's airways. Alternatively, a manual registration technique may be employed by navigating the bronchoscope 50 with the EM sensor 94 to pre-specified locations in the lungs of the patient P, and manually correlating the images from the bronchoscope to the model data of the three-dimensional model.

The methods described herein may be used in conjunction with robotic systems such that robotic actuators drive the catheter 96 or bronchoscope 50 towards and proximate to the target.

FIG. 2 is a perspective view of an end portion of a deflectable catheter (EWC) 200 in accordance with the disclosure. The catheter 200 includes an inner tubular structure 215 and outer tubular structures 201, 202. The inner tubular structure 215 may be made of Polytetrafluoroethylene (PTFE) or any other flexible material suitable for providing a base structure on which other components of the catheter 200, e.g., the pull ring 211, can be attached or disposed. The catheter 200 may include an assembly liner 205 on the inner surface of the inner tubular structure 215. The outer tubular structures 201, 202 may be a thermoplastic elastomer made of flexible polyether and rigid polyamide. The outer tubular structure 202 may be less rigid than the outer tubular structure 201 to allow more flexibility of the catheter 200 proximally from the distal end portion of the catheter 200.

The catheter 200 also includes a pull ring 211, which is attached to the outer surface of the inner tubular structure 215, and a pull wire 212 is attached to the outer surface of the pull ring 211. The pull wire 212 may be laser welded to the outer surface of the pull ring. The pull wire 212 may be attached to the pull ring 211 using any other method suitable for ensuring that a pull force repeatedly applied to the pull wire 212 does not cause the pull wire 212 to separate from the pull ring 211. During manufacturing of the catheter 200, the distal end portion of the pull wire 212 may be attached to the pull ring 211 before the pull ring 211 is slid onto the inner tubular structure 201.

The catheter 200 also includes a pull wire lumen 206 in which the pull wire 212 is disposed and allows the pull wire 212 to slide within the pull wire lumen 206. The pull wire lumen 206 may be made of Polytetrafluoroethylene (PTFE) or any other flexible material suitable for protecting the pull wire 212. The catheter 200 also includes sensor wire 213 a first portion of which is wrapped around the distal end portion of the inner tubular structure 215 and a second portion of which extends in a proximal direction through the catheter 200 to an electrical connector (not shown). The electrical connector may connect to the tracking system 114 shown in FIG. 1. Alternatively, the first winding portion of the sensor wire 213 may be a separate component from the second portion of the sensor wire 213. During manufacturing, the first portion of the sensor wire 213 may be attached to the second portion of the sensor wire 213 using a method suitable for robustly connecting wires together so that the wires do not disconnect while the distal end portion of the catheter 200 is being deflected. The catheter 200 may also include a sensor wire lumen 207, which contains the second portion of the sensor wire 213. The sensor wire lumen 207 may be made of a material that shields that second portion of the sensor wire 213 from electromagnetic interference. The material may include a metal, metal alloy, or conductive polymer that is flexible and robust against repeated flexing.

The catheter 200 also includes braid wire 208 wrapped around a proximal portion of the inner tubular structure 215. The braid wire 208 may be disposed between the inner tubular structure 215, and the pull wire lumen 206 and the sensor wire lumen 207. The braid wire pattern may include paired wires having a diamond pattern as illustrated in FIG. 2. The catheter 200 also includes coil wire 210 disposed along a proximal portion of the catheter 200.

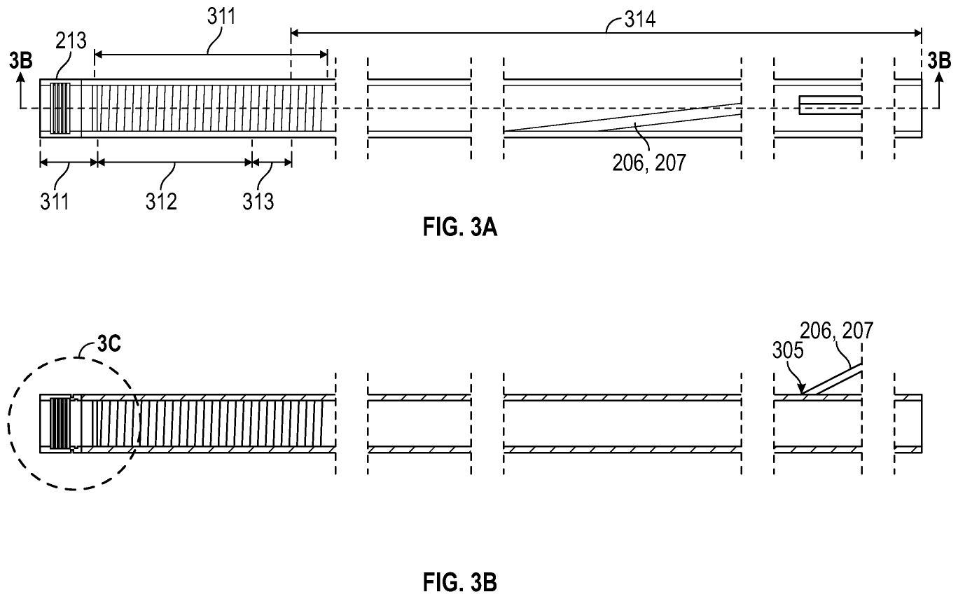

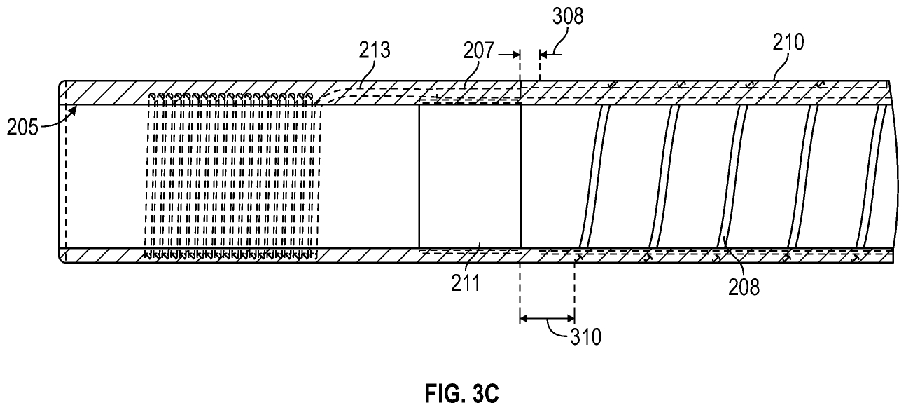

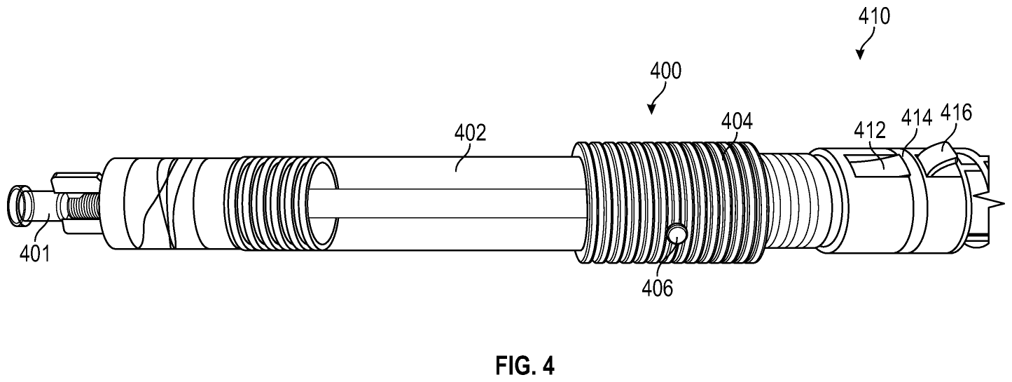

Turning to FIGS. 3A-3C, the length of the catheter 200 is shown. The outer tubular structure may include four extrusion portions 311-314, which may be formed from thermoplastic elastomers (TPEs). Extrusion portions 311-313 may be made of flexible polyether and rigid polyamide. Extrusion portion 312 may be less rigid than extrusion portions 311, 313 to enable bending of extrusion portion 312. Extrusion portion 314 may be formed from a high viscosity polyamide that is resistant to body fluids and is toxicologically safe. As shown in FIG. 3C, the distance 310 between the distal end of the coil wire 310 and the pull ring 211 may be greater that the distance 308 between the distal end of the braided wire 210 and the pull ring 211. The pull wire lumen 206 and the sensor wire lumen 207 may be disposed along a length of the catheter 200 between the inner tubular structure 215 and the extrusion portions 311-314, but may exit through a slot 305 in extrusion portion 314.

FIG. 4 shows an example of a handle assembly 400, which may be used to manipulate or operate the catheter 200. The handle assembly 400 includes a housing 402 and a tip 401, through which with the catheter 200 passes. The handle assembly 400 also includes a handle portion 404, which may be held by a clinician or a structure for holding the handle assembly 400. In aspects, the handle portion 404 is shaped such that a clinician or a structure can maintain the position of the handle assembly 400 when the catheter 200 is operated. The handle assembly 400 also includes a pull handle assembly 410, which is connected to a proximal end portion of the pull wire 207, and which includes a user-operated pull handle 416. When the pull handle 416 is pulled in a proximal direction, the pull wire 212 causes the distal end portion of the catheter 200 to deflect. The pull handle 416 may be shaped such that the pull handle 416 may be operated by a clinician's thumb. Alternatively, the pull handle 416 may be shaped such that the pull handle 416 may be operated by the pinching action of a clinician's forefinger and thumb.

The pull handle assembly 410 also includes a locking portion 414 and a detent portion 412, which is configured to engage with the locking portion 414. As shown in FIG. 4, the locking portion 414 may be formed by a portion of the housing 402. The detent portion 412 may include ridges that engage with a proximal portion of the locking portion 414 to hold the pull handle assembly 410 in a fixed position. The detent portion 412 is disengaged from the locking portion 414 when the clinician depresses the pull handle 416. In aspects, the detent portion 412 may include one or more detents or ridges that engage with the locking portion 414. While a pull handle assembly 410 utilizing detents is described, the pull hand assembly 410 may incorporate other mechanisms for holding the distal end portion of the catheter 200 at a fixed orientation, for example a detent (not shown) formed on the pull handle assembly 414 may engage and be released by application of pressure to the button 406 on the side of the handle assembly 400.

As will be appreciated the pull handle 416, which engages the pull wire 207 can be implemented in a number of ways without departing from the scope of the disclosure. For example, the pull handle 416 may be connected to a drum to which the pull wire 207 is connected. Translation of the lever winds the pull wire 207 over the drum to shorten its effective length and effectuate articulation of the catheter 200. Similarly in place of the drum, a bell crank can be employed. The bell crank, as is known, has a fixed pivot point and two moving pivot points. The pull wire 207 is connected to one of the moving pivot points, and the pull handle 416 may be attached to the second. Application of pressure to the pull handle 416 in a first direction causes the bell crank to rotate about the fixed pivot point and to pull the pull wire 207, connected to the second moving pivot point in a first direction to articulate the catheter 200. Release or application of force to the pull handle 416 in a second direction allows the second moving point to move in a second direction and to relax the articulation.

Additionally or alternatively, the pull wire 207 may be attached to a threaded collar that is connected to a lead screw. Rotation of the leadscrew in a first direction causes the leadscrew to engage the threads of the collar and move the collar in a first direction. This movement in the first direction engages the pull wire 207 to articulate the catheter 200. Rotation of the leadscrew in a second direction moves the collar in a second direction and to relax the articulation. The use of a collar and lead screw is particularly amenable to motorization such that there is no direct user application of mechanical force to the pull wire 207, but rather force is applied via an electric or pneumatic motor to achieve the desired articulation. Other methods and systems for acting on the pull wire 207 and effectuating articulation may be employed without departing from the scope of the disclosure.

While detailed embodiments are disclosed herein, the disclosed embodiments are merely examples of the disclosure, which may be embodied in various forms and aspects. For example, embodiments of an electromagnetic navigation system, which utilize the deflectable catheter and the EM sensor wire disposed on the distal end portion of the catheter, are disclosed herein; however, the deflectable catheter may be applied to other navigation or tracking systems or methods known to those skilled in the art. Therefore, specific structural and functional details disclosed herein are not to be interpreted as limiting, but merely as a basis for the claims and as a representative basis for teaching one skilled in the art to variously employ the disclosure in virtually any appropriately detailed structure.

What is claimed is:

1. A catheter navigation system comprising:
an electromagnetic field generator configured to generate an electromagnetic field;
a deflectable catheter including:
a tubular structure configured to receive and guide a surgical tool;
a sensor disposed around an end portion of the tubular structure and configured to sense the electromagnetic field;
a pull wire assembly coupled to the end portion of the tubular structure and configured to cause a distal portion of the tubular structure to deflect when the pull wire assembly is operated; and
a coil disposed around a portion of the tubular structure configured to restrain the pull wire; and
a tracking system electrically coupled to the electromagnetic field generator and the sensor wire, the tracking system configured to determine a location of the sensor wire based on the electromagnetic field sensed by the sensor wire.

2. The catheter navigation system of claim 1, wherein the pull wire assembly includes a pull ring fixedly coupled to the end portion of the tubular structure and a pull wire coupled to the pull ring.

3. The catheter navigation system of claim 2, wherein the sensor is positioned distal from the pull ring.

4. The catheter navigation system of claim 2, wherein the catheter further includes braided wire disposed around a portion of the tubular structure.

5. The catheter navigation system of claim 4, wherein the coil is disposed around the braided wire and the pull wire.

6. The catheter navigation system of claim 5, wherein the pull wire assembly further includes a lumen disposed between the coil and the braided wire, and configured to receive the pull wire.

7. The catheter navigation system of claim 5, wherein the sensor includes a wire disposed along a portion of the length of the catheter between the braided wire and the coil.

8. The catheter navigation system of claim 5, wherein the catheter further includes a lumen disposed between the coil and the braided wire, and configured to receive the wire.

9. A deflectable catheter comprising:
a tubular structure configured to receive and guide a surgical tool;
a sensor disposed around an end portion of the tubular structure and configured to sense an electromagnetic field;
a pull wire assembly coupled to the end portion of the tubular structure and configured to cause a distal portion of the tubular structure to deflect when the pull wire assembly is operated; and
a coil wire disposed around a portion of the tubular structure and configured to restrain the pull wire.

10. The deflectable catheter of claim 9, wherein the pull wire assembly includes a pull ring fixedly coupled to the end portion of the tubular structure and a pull wire coupled to the pull ring.

11. The deflectable catheter of claim 10, wherein the pull wire engages the pull ring to deflect to catheter.

12. The deflectable catheter of claim 10, wherein the sensor is positioned distal from the pull ring.

13. The deflectable catheter of claim 10, wherein the catheter further includes braided wire disposed around a portion of the tubular structure.

14. The deflectable catheter of claim 10, wherein the coil is disposed around the braided wire and the pull wire.

15. The deflectable catheter of claim 14, wherein the pull wire assembly further includes a lumen disposed between the coil and the braided wire, and configured to receive the pull wire.

16. The deflectable catheter of claim 14, further comprising a wire coupled to the sensor and disposed along a portion of the length of the catheter between the braided wire and the coil.

17. The deflectable catheter of claim 14, further comprising a lumen disposed between the coil and the braided wire, and configured to receive the sensor wire.

18. A catheter guide assembly comprising:
a catheter including:
a tubular structure configured to receive and guide a surgical tool;
a sensor disposed around an end portion of the tubular structure and configured to sense the electromagnetic field;
a pull wire assembly coupled to the end portion of the tubular structure and configured to cause a distal portion of the tubular structure to deflect when the pull wire assembly is operated; and
a coil wire disposed around a portion of the tubular structure configured to retain the pull wire; and
a handle assembly including:
a handle; and
a manipulator coupled to the pull wire and enabling a user to deflect the distal end portion of the catheter.

19. The catheter guide assembly of claim 18, further comprising a second tubular structure enclosing the catheter.

20. The catheter guide assembly of claim 18, wherein the manipulator includes a slider having detents that engage with a locking member to maintain the distal end portion of the catheter in a deflected state.
